(19) United States
(12) Patent Application Publication
(10) Pub. No.: US 2016/0051394 A1
(43) Pub. Date: Feb. 25, 2016

(54) A DEVICE FOR DIRECTING OUTPUT FROM A STOMA

(71) Applicant: PERISENSE APS, Hillerød (DK)

(72) Inventor: Brian NIELSEN, Gørløse (DK)

(21) Appl. No.: 14/888,521

(22) PCT Filed: May 5, 2014
(86) PCT No.: PCT/EP2014/059142

§ 371 Date: Nov. 2, 2015

(30) Foreign Application Priority Data
May 3, 2013 (DK) PA201300262

(51) Int. Cl. A61F 5/445

(52) U.S. Cl. CPC A61F 5/445

(57) ABSTRACT

A device for directing output from a stoma, the device comprising at least one elastic element, said elastic element comprising a first portion for receiving and contacting the stoma and second portion for attaching the elastic element to the device; an attachment element for attaching the device to the human being, the 5 attachment element comprising a first surface and a stretching element for stretching at least a portion of the elastic element.

### FIELD OF THE INVENTION

The present invention relates to a device for directing output from a stoma and to a system for directing and collecting output from a stoma. The invention also relates to methods of providing the device and the system.

### BACKGROUND OF THE INVENTION

A known problem with ostomy bags is that there are frequent leakages between the skin surrounding the stoma and the adhesive in the attachment of the ostomy bag.

Many have tried to solve these issues by improving the flexibility of the adhesive, increasing the absorbent capacity of the adhesive and by cutting mechanisms trying to fit the adhesive's to the shape of the stoma.

Furthermore some inventions have been made trying to seal the connection between the stoma and the waste container. However some ostomies have a shape where it is difficult to create such a seal and maintain it during wear time.

It is therefore desirable to invent a faeces collecting system, such as an ostomy bag, that ensures that a seal is made between the stoma and the faeces collecting system; and a mechanism or design ensuring that the seal is maintained during the wear time of the faeces collecting system.

U.S. Pat. No. 3,878,847 tries to solve this problem by introducing an integrated elastic film or foam with a hole slightly smaller than the diameter of the stoma. The interface between the stoma and the film will be sealed by the joint elasticity of the stoma and the elasticity of the film, but the resulting structure is rather complex.

WO2010034966 tries to build a seal between the outer surface of the stoma and a container by providing an elastic shape with a hole slightly smaller than the diameter of the stoma.

However many stoma patients still fail to obtain an effective seal and are consequently struggling with damaged or irritated skin and leakages.

It is therefore desirable to provide a kit of parts for collecting output from a human being, wherein the kit of parts provide an effective seal avoiding output from a protruding shape such as a stoma to contact the human beings skin.

### SUMMARY OF THE INVENTION

In a first aspect, the present invention relates to a device for directing output from a stoma, the device comprising:
at least one elastic element, said elastic element comprising a first portion for receiving and contacting the stoma and second portion for attaching the elastic element to the device;
an attachment element for attaching the device to the human being, the attachment element comprising a first surface; and
a stretching element for stretching at least a portion of the elastic element.

In a second aspect, the present invention relates to a system for directing and collecting output from a stoma, the system comprising:
a device according to the first aspect of the invention; and
a suitable container for collecting the output from the stoma.

In a third aspect, the present invention relates to a method for providing a device for directing output from a stoma of a human being, the method comprising the steps of:
providing an elastic element with a first and second portion;
providing a stretching element;
providing an attachment element;
expanding the second portion of the elastic element; and
connecting the second portion of the elastic element to the stretching element.

In a fourth aspect, the present invention relates to a method for providing a system for directing and collecting output from a stoma of a human being, the method comprising the steps of:
providing a device according to the third aspect of the present invention;
providing a suitable container for collecting output from the stoma; and
attaching the suitable container to the device.

### BRIEF DESCRIPTION OF THE DRAWINGS

FIG. 1a to 1d disclose different views of an elastic element according to the present invention.

FIGS. 2a and 2b disclose different views of a device for directing output from a stoma according to the present invention.

FIGS. 3a to 3d disclose different views of an elastic element according to the present invention.

FIGS. 4a and 4b disclose different views of a device for directing output from a stoma according to the present invention.

FIGS. 5a to 6b disclose different views of a device for directing output from a stoma and an adjusting element according to the present invention.

FIGS. 7 and 8 disclose cross sectional views of systems for directing and receiving output from a stoma according to the present invention, where the systems are applied to a stoma.

FIGS. 9a to 9d disclose different views of an adjusting element according to the present invention.

FIGS. 10 and 11 disclose cross sectional views of different devices for directing output from a stoma according to the present invention.

FIGS. 12a to 12c disclose cross sectional views of a device for directing output from a stoma according to the present invention, wherein the device is disclosed prior to application, during application and during wear-time on the human beings stoma.

FIGS. 13a and 13b disclose cross sectional views of a device according to the present invention, wherein the adjusting element is in two different positions.

FIG. 14 shows a schematic cross section view of an elastic element.

FIG. 15 shows the elastic element of FIG. 14 stretched.

FIG. 16 shows the elastic element of FIG. 14 attached to a stretching element.

FIG. 17-19 show views of a concreted embodiment of a stretching element according to the invention.

FIGS. 20-21 schematically show in cross section two different embodiments of a device according to the invention.

FIGS. 22-25 show four different embodiments of a stretching element with integrated adjusting element.

### DETAILED DESCRIPTION OF THE INVENTION

The first aspect of the present invention relates to a device for directing output from a stoma, the device comprising:
at least one elastic element, said elastic element comprising a first portion for receiving and contacting the stoma and second portion for attaching the elastic element to the device;
an attachment element for attaching the device to the human being, the attachment element comprising a first surface; and
a stretching element for stretching at least a portion of the elastic element.

In the context of the present invention the term “device” shall be understood as a physical unit, the unit being a system, a part of a system or a separate product intended to contact a stoma (ileo, colo or urostoma). The device may be arranged around the stoma. It could also be noted that the device acts as a form of “sealing element” which forms a seal between the stoma of a human being.

In the context of the present invention the term “elastic element” shall be understood as a physical element, the physical element comprising an elastic material that forms a contact to the outer surface of the stoma during the wear time of the device. The elastic element has a hole of any shape adapted to receive the stoma.

In the context of the present invention the term “attachment element” shall be understood as a physical element being adapted to attach the device to the human being by any means. Such means include belt structures, adhesives and the like.

In the context of the present invention the term “first surface” shall be understood as a surface on the attachment element. The first surface is, when applied on the human being, the portion of the attachment element either in direct contact with or being closest to the peri-stomal skin. The first surface is defined to be essentially parallel to the peri-stomal skin. The first surface may have an irregular shape for example a pattern.

In the context of the present invention the term “stretching element” shall be understood as a physical element having a larger circumference than the first portion of the elastic element.

The first portion of the elastic element has a hole for receiving a stoma. The first portion of the elastic element is intended to contact the outer surface of the stoma and seal against it. The seal between the stoma and the first portion is achieved by the combined elasticity of the stoma and the elasticity of the first portion of the elastic element. It is important to use a material that is so elastic, that it on the one hand does not harm the stoma and on the other hand creates a good seal against the outer surface of the stoma. The elastic element should also hinder the stoma output to contact the peri-stomal skin. The less elastic the elastic element is the more important becomes the size of the hole in the elastic element and its fit to the diameter of the stoma, while with increased elasticity of the elastic element, the allowed diameter difference between the hole in the elastic element and the outer diameter of the stoma increases.

In one embodiment of the first aspect of the present invention the hole in the elastic element has a diameter from of 8 to 50 mm.

In one embodiment of the first aspect of the present invention the hole is between 1 and 20 mm smaller in diameter than the stoma it is intended to contact.

In one embodiment of the first aspect of the present invention, the first portion of the elastic element is made from an elastic film material between 0.02 and 2 mm thick. It is preferred, that the first portion of the elastic element is between 0.05 and 1 mm thick. It is more preferred that the first portion of the elastic element is between 0.1 and 0.5 mm thick and it is most preferred that the elastic film is between 0.1 and 0.3 mm thick.

In one embodiment of the first aspect of the present invention, the elastic element is of a ring formed shape, for example a flat, planar disc shaped element similar to a CD

In one embodiment of the first aspect of the present invention, the elastic element is of a tubular shape.

In one embodiment of the first aspect of the present invention, the first portion of the elastic element is of a tubular shape.

In the context of the present invention the term “tubular shape” shall be understood as a shape formed like a pipe, a cylinder, a truncated cone, a diamond shape, a cylindrical shape with varying cross sectional diameters, an oval cylinder, combinations of the above or other cylindrically shaped structures with regular or irregular cross sectional geometries.

In one embodiment of the first aspect of the present invention, the elastic element comprises a tubular shape with a flange portion.

In one embodiment of the first aspect of the present invention, the elastic element comprises a tubular area and a flange area, wherein the first portion of the elastic element is located on the tubular area and the second portion of the elastic element is located on the flange area.

The second portion of the elastic element is adapted to attach the elastic element to the rest of the device. The second portion is the part of the elastic element that engages with at least one other element of the device. Such engaging means may be achieved by using an adhesive, a weld, glue, a mechanical fastener or the like.

In one embodiment, the second portion of the elastic element is mechanically fastened onto another element of the device.

In one embodiment, the second portion of the elastic element is welded onto another element of the device.

In one embodiment of the first aspect of the present invention the second portion of the elastic element is attached to the attachment element.

In one embodiment of the first aspect of the present invention the second portion of the elastic element is attached to the stretching element.

As mentioned earlier, the elasticity and softness of the material used to make the elastic element is important. The modulus, elongation and shore OO hardness are expressions of such elasticity and softness.

In the context of the present invention, the term “modulus 200%” describes the stress (measured in MPa) required to elongate a sample of given dimensions 200%, measured according to the standard test method ASTM D638.

In one embodiment, the elastic element is made of a material having a modulus 200% between 0.01 and 2.00, between 0.05 and 1.50, between 0.05 and 1.0 or between 0.07 and 0.8 MPa.

In the context of the present invention the term “elongation” shall be understood as the percentage the material is capable of being expanded before it breaks (test method ASTM 412, elongation (%) @ break).

In one embodiment, the elastic element is made of a material having an elongation between 150 and 2000%, between 250 and 1500%, between 500 and 1200% or between 800 and 1500%.

In the context of the present invention the term “shore OO hardness” shall be understood as the hardness of the material measured by test method ASTM 2240 (at 23° C., 10 sec.).

In one embodiment of the first aspect of the present invention, the elastic element is made of a material having a shore OO hardness from 20 to 90, from 25 to 85, from 30 to 80 or from 32 to 72. In one embodiment of the first aspect of the present invention, the elastic element is made of a material having a shore OO hardness from 60 to 90.

In the case where the shore OO value of the elastic element is relatively low, a too thin material may easily be damaged, and since the shore OO value is low, a thicker material may be used. Therefore the thickness of the elastic element may increase with decreasing shore OO value.

In one embodiment of the first aspect of the present invention, the wall thickness of the first portion of the elastic element is from 0.05 to 0.50 mm thick or from 0.1 to 1.0 mm thick. It is preferred that the first portion of the elastic element has a wall thickness of from 0.1 to 0.4 mm.

As a portion of the elastic element may be stored prior to use in a stretched condition, it may be desirable to use materials with high shape-memory characteristics. Such materials include cross-linked polymers such as some polyurethanes and silicones. Furthermore materials with a higher shore OO value tend to have better shape memory.

In one embodiment of the first aspect of the present invention, the elastic element is made of a cross-linked polymer. It is preferred, that the elastic element is made of a silicone material.

Materials used to produce the elastic element include thermoplastic elastomeric materials similar to the Versaflex™ grades, for example Versaflex™ 2000 or Nusil silicone grades, for example Nusil Med-4901 or Nusil Med-4020.

In order to create an effective contact between the elastic element and the stoma, it is important that the hole in the first portion of the elastic element is smaller than the diameter of the stoma.

In one embodiment of the first aspect of the present invention, the hole in the first portion of the elastic element is smaller than the diameter of the outer surface of the stoma at the sealing point. It is preferred that the diameter of the hole in the elastic element is at least 4 mm smaller than the diameter of the stoma.

In one embodiment of the first aspect of the present invention, the diameter of the hole in the first portion of the elastic element is from 8 to 35 mm. It is preferred that the diameter of the hole is from 14 to 28 mm.

In one embodiment of the first aspect of the present invention the first portion of the elastic element comprises a truncated cone shape.

In one embodiment of the first aspect of the present invention, the elastic element is essentially planar.

In order to get a larger contact surface between the stoma and the first portion of the elastic element, the first portion of the elastic element may be shaped like a cylinder.

In one embodiment of the first aspect of the present invention, the first portion of the elastic element comprises a cylindrical shape. In one embodiment of the first aspect of the present invention, the cylindrical shape of the first portion of the elastic element is from 2 to 50 mm long. It is preferred that the cylindrical shape of the first portion of the elastic element is from 5 to 20 mm long. It is more preferred that the cylindrical shape of the first portion of the elastic element is from 4 to 16 mm long.

The device for directing output from a stoma comprises an attachment element. The attachment element is used to attach the device to the human being. In one embodiment of the first aspect of the present invention, the attachment element comprises a belt structure.

In one embodiment of the first aspect of the present invention, the attachment element comprises a skin friendly adhesive.

Such an adhesive may be any skin friendly adhesive. Some examples of suitable adhesives include polyacrylates, polyurethanes, hydrocolloids and silicone adhesives. Silicone adhesives are known to be particularly skin friendly.

In one embodiment of the first aspect of the present invention, the attachment element comprises a silicone adhesive.

If stoma output or liquid from the mucosa should contact the peri-stomal skin, it may be an advantage, that the adhesive is absorbent.

In one embodiment of the first aspect of the present invention, the attachment element comprises a liquid absorbent adhesive.

In one embodiment of the first aspect of the present invention, the liquid absorbent adhesive comprises cross-linked Carboxy Methyl Cellulose.

Furthermore, to protect the peri-stomal skin, it may be advantageous, that the adhesive is distributed on the attaching element in a pattern.

In one embodiment of the first aspect of the present invention, the attachment element comprises an adhesive, wherein the adhesive is present in a pattern.

It may be desirable to have an attachment element with at least a portion being similarly semi-rigid in order to obtain good adherence.

In one embodiment of the first aspect of the present invention, the device for directing output from a stoma comprises a semi-rigid attachment element.

The device for directing output from a stoma comprises a stretching element for stretching at least a portion of the elastic element. It should be noted that the purpose of the stretching element is to maintain a portion of the elastic element in a stretched configuration. The stretching element can in some embodiments both actively stretch the elastic element and then maintain it in the stretched position, while in other embodiments, an external force is used to stretch the elastic element whereafter the stretching element just maintains the elastic element in its stretched configuration. In other words, the stretching element in some embodiments is an active element and in other embodiments is just a static element.

The stretching of a portion of the elastic element is used to get an engagement of the first portion of the elastic element on the outer surface of the stoma close to the peri-stomal skin and not too outwardly on the stoma. An outwards engagement between the surface of the stoma and the first portion of the elastic element may lead to a leakage of stoma output as the peristaltic forces of the stoma may cause disengagement between the two. The stretched portion of the elastic element may be close to or directly adjacent to the first portion of the elastic element in order to support the position of the engagement.

The stretching element stretches a portion of the elastic element. The more stretched the portion of the elastic element is, the better support is provided to the engagement of the first portion of the elastic element on the surface of the stoma. On the other hand, the more stretched the portion of the elastic element is, the larger a force is used to stretch the portion. Such force may put restrictions on the design of the device, for example it will influence the rigidity of the device.

In one embodiment of the first aspect of the present invention, the stretching element stretches the diameter of a portion of the elastic element from 10 to 500%. It is preferred that the stretching element stretches the diameter of a portion of the elastic element from 40 to 300%. It is more preferred that the stretching element stretches the diameter of a portion of the elastic element from 60 to 200%.

In one embodiment of the first aspect of the present invention, the device is characterised by the circumference of the stretched portion of the elastic element being at least stretched to a circumference 40% larger than the circumference of the portion when not stretched.

In one embodiment of the first aspect of the present invention, the device is characterised by the circumference of the stretched portion of the elastic element being at least stretched to a circumference from 30 to 150% larger than the circumference of the portion when not stretched.

In one embodiment of the first aspect of the present invention, the device is characterised by the circumference of the stretched portion of the elastic element being at least stretched to a circumference from 20 to 60% larger than the circumference of the portion when not stretched.

In one embodiment of the first aspect of the present invention, the stretching element stretches the second portion of the elastic element.

The device for directing output from a stoma may be coupled to a container for receiving and for a period of time storing the output from the stoma.

In one embodiment of the first aspect of the present invention, the device for directing output from a stoma comprises a connecting element for connecting the device to a container.

In the context of the present invention the term “container” shall be understood as a physical element of any shape with an enclosed volume. The design includes bags, bottles and the like, and the element may have an outlet for regularly emptying the enclosed volume.

In one embodiment of the first aspect of the present invention, the connecting element for connecting the device to a container is adapted to reversibly connect and disconnect from the container.

Some humans have a relatively short protruding length of their stoma. Sometimes, the stoma is even planar to the peri-stomal skin. On such humans it may be desirable to push the peri-stomal skin inwards in relation to the normal surface. It may also be desirable to move the stoma-contacting portion of the elastic element to a more inwards position to reduce risk of leakages.

In one embodiment of the first aspect of the present invention, the device comprises an adjusting element for adjusting the position of at least a portion of the elastic element relative to the attachment element in at least one direction.

It should be noted that in the context of the current specification, the adjusting element should be understood as an element which adjusts the position of the elastic element relative to the position without an adjusting element. In certain embodiments the adjusting element is fixed in location and its own position cannot be “adjusted”. In other embodiments the adjusting element is arranged to be moveable such that its own position can be adjusted. However, both fixed and moveable adjustment elements are included within the scope of the current invention.

In one embodiment of the first aspect of the present invention, the adjusting element is adapted to adjust the position of at least a portion of the elastic element relative to the attachment element in at least the direction perpendicular to the first surface of the attachment element.

In one embodiment of the first aspect of the present invention, the adjusting element is adapted to move the position of at least a portion of the elastic element relative to the attachment element in the direction perpendicular to the first surface to a new position between 1 and 40 mm more inwards than the attachment element. A direction perpendicular and more inwards can be understood as being “normal” to the first surface.

In one embodiment of the first aspect of the present invention, the adjusting element comprises an anchoring portion and a movable portion.

In the context of the present invention the term “move to a more inwards position” shall be understood as an adductive movement.

In the context of the present invention the term “anchoring portion” shall be understood as a physical shape adapted to attach the moveable portion of the adjusting element to the attaching element or the device.

In the context of the present invention the term “movable portion” shall be understood as a physical shape adapted to be moved adductively in relation to its first position.

In one embodiment of the first aspect of the present invention, the movable portion of the adjusting element has at least a first disengaged position and a second engaged position.

In the context of the present invention the term “disengaged position” shall be understood as a position where the adjusting element does not move at least a portion of the elastic element relative to the attachment element in the direction perpendicular to the first surface.

In the context of the present invention the term “engaged position” shall be understood as a position where the adjusting element moves at least a portion of the elastic element relative to the attachment element in the direction perpendicular to the first surface.

In one embodiment of the first aspect of the present invention, the movable portion of the adjusting element is adapted to be positioned in at least 2 different positions relative to the anchoring portion.

In one embodiment of the first aspect of the present invention, the movable portion of the adjusting element is adapted to be positioned in at least 3 different positions relative to the anchoring portion.

In one embodiment of the first aspect of the present invention, the device comprises a first locking mechanism for locking the position of the movable portion of the adjusting element.

In the context of the present invention the term “first locking mechanism” shall be understood as a physical shape adapted to fasten the position of the movable portion. Such fastening may or may not be unfastened. The position of the moveable portion may be completely locked or just fastened so that an increased force may move the movable portion.

In one embodiment of the first aspect of the present invention, the locking mechanism comprises a manual unlocking mechanism.

In one embodiment of the first aspect of the present invention, the locking mechanism comprises an automatic unlocking mechanism.

In one embodiment of the first aspect of the present invention, the locking mechanism is automatically unlocked when the outwards force from the peri-stomal skin and tissue exceeds a threshold value.

The elastic element may contact the peri-stomal skin, depending on the design of the device. Further, the elastic element may have reduced vapour transmission rates and therefore cause some degree of discomfort. Consequently, depending on the design of the device and depending on the material used as elastic element it may be desirable to have a skin contacting element. The skin contacting element may reduce the skin contacting surface of the elastic element.

In one embodiment of the first aspect of the present invention, the device comprises a skin contacting element.

In the context of the present invention the term “the skin contacting element” shall be understood as a physical shape or material not adhering to the peri-stomal skin, preferably made out of a fibrous structure.

In one embodiment of the first aspect of the present invention, the skin contacting element comprises a fibrous structure.

In one embodiment of the first aspect of the present invention, the skin contacting element is essentially made out of a fibrous structure.

In one embodiment of the first aspect of the present invention, the skin contacting element comprises a non-woven fibrous structure.

In one embodiment of the first aspect of the present invention, the skin contacting element comprises a woven fibrous structure.

In one embodiment of the first aspect of the present invention, the skin contacting element has a liquid absorption capacity.

In one embodiment of the first aspect of the present invention, the skin contacting element has absorption capacity from 0.03 to 0.7 g/cm² of 0.9% saline water solution.

In one embodiment of the first aspect of the present invention, the skin contacting element has absorption capacity from 0.06 to 0.5 g/cm² of 0.9% saline water solution.

In one embodiment of the first aspect of the present invention, the skin contacting element comprises liquid absorbent fibres.

In one embodiment of the first aspect of the present invention, the skin contacting element comprises hydrophobic fibres.

It may be desirable to include the device in a system or a kit of parts, the system being capable of both directing and collecting the output from the stoma. Such collected output may be stored in a container for a period of time (from 1-2 hours to up to more than one day). It may be desirable to allow the container to be emptied without removing the container from the device.

A second aspect of the present invention relates to a system for directing and collecting output from a stoma, the system comprising:
a device according to the first aspect of the invention; and
a suitable container for collecting the output from the stoma.

In one embodiment of the second aspect of the present invention, the system comprises an emptying mechanism for emptying the container.

In one embodiment of the second aspect of the present invention, the suitable container is a plastic bag.

In one embodiment of the second aspect of the present invention, the suitable container is remote from the attachment element of the device.

In one embodiment of the second aspect of the present invention, the suitable container is connected to the elastic element.

In one embodiment of the second aspect of the present invention, the suitable container is connected to the elastic element in such a way that the interior of the tubular portion of the elastic element is in fluid communication with the interior of the suitable container.

A third aspect of the present invention relates to a method for providing a device for directing output from a stoma of a human being, the method comprising the steps of:
providing an elastic element with a first and second portion;
providing a stretching element;
providing an attachment element;
expanding the second portion of the elastic element; and
connecting the second portion of the elastic element to the stretching element to maintain a portion of the elastic element in a stretched configuration.

A fourth aspect of the present invention relates to a method for providing a system for directing and collecting output from a stoma of a human being, the method comprising the steps of:
providing a device according to the third aspect of the present invention;
providing a suitable container for collecting output from the stoma; and
attaching the suitable container to the device.

It will be appreciated that any combination of features and elements of the first, second, third, and fourth aspect may be combined in any suitable manner.

### DETAILED DESCRIPTION OF THE DRAWINGS

The below list of drawings of examples of the present invention should in no way limit the scope of the invention, but be seen as a few of many different designs of a device according to the present invention.

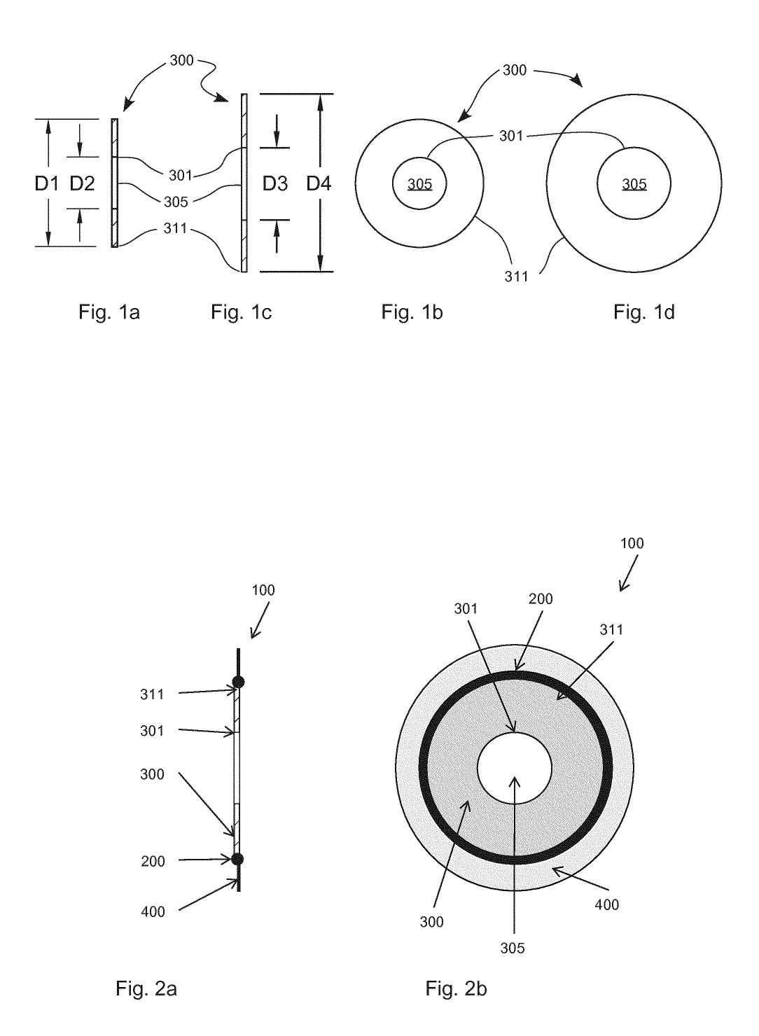

FIG. 1a-1d disclose views of a first embodiment of an elastic element (300) of a device for directing and receiving output from a stoma. In this first embodiment, the elastic element is of a planar disc formed shape.

FIG. 1a discloses a cross sectional view of the first embodiment of an elastic element in an unstretched position.

FIG. 1b discloses a frontal view of the elastic element (300) in its unstretched position. The elastic element has a first portion (301) for receiving the stoma and a second portion (311) for connecting the element to another element of the device. The first portion (301) is the circumference of a hole (305) adapted to receive the stoma of the human being. The second portion (311) of the elastic element is the edge of the disc formed element. In FIGS. 1a and 1b, the second portion of the elastic element is not stretched.

FIG. 1c discloses a cross sectional view of the elastic element from FIG. 1a-b, comprising a first portion (301) and a second portion (311), but now the second portion (311) has been stretched. As can be seen the inner diameter of the first portion has been stretched from the previous diameter D1 to the new diameter D3. Likewise the outer diameter of the second portion has been stretched from the previous diameter D2 to the new diameter D4.

Similarly FIG. 1d discloses a frontal view of the elastic element from FIG. 1a-b, comprising a first portion (301) and a second portion (311), where the second portion (311) has been stretched.

FIG. 2a-2b disclose views of an example of a device (100) according to the present invention, the device comprising an elastic element (300) similar to the elastic element described in with regards to FIGS. 1a-d, the elastic element being connected to a stretching element (200) and a device comprising an attachment element (400) for attaching the device to a human beings peri-stomal skin.

FIG. 2a discloses a cross sectional view of a device (100), comprising an elastic element (300) with a first portion (301) for receiving and contacting the stoma and a second portion (311) for connecting the elastic element to the device. The device also comprises an attachment element (400) for attaching the device to a human being. In this example, the attaching element (400) is a skin friendly adhesive coated onto a disc-formed film, which is welded to the outer circumference of the ring-formed stretching element (200).

FIG. 2b discloses a frontal view of the device (100), comprising an elastic element (300) with a first portion (301) and a second portion (311). The disc formed elastic element's first portion (301) is at the circumference of the hole (305) for receiving the stoma. The device comprises a ring-formed stretching element (200) for stretching at least a portion of the elastic element. In this example, the second portion of the elastic element is first stretched manually and then glued to the stretching element.

It should be noted that the elastic element is arranged such that after it has been stretched, the hole in the first portion of the elastic element is still slightly smaller than the stoma.

In another embodiment (not shown), the second portion of the elastic element is welded to the stretching element after which the elastic element is treated, for example heat treated, to cause the material of the elastic element to shrink, thereby causing the elastic element to stretch and thereby introduce pre stress into the elastic element.

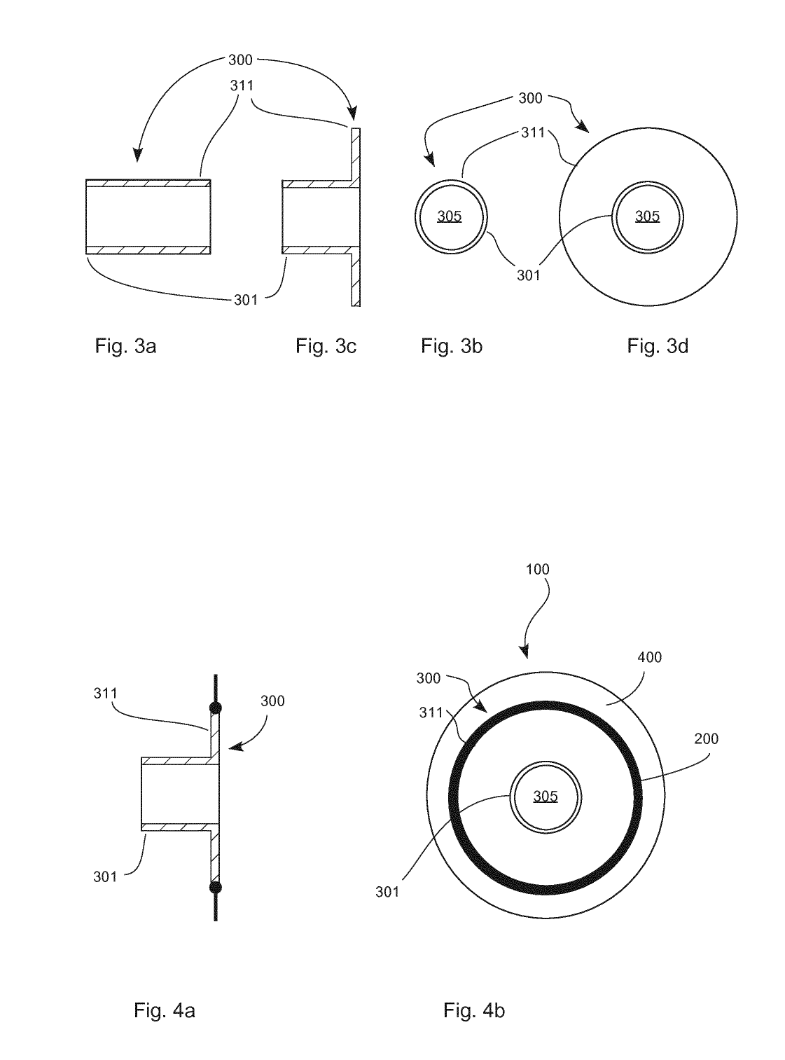

FIGS. 3a to 4b disclose views of an elastic element and a device similar to the embodiment shown in FIGS. 1a to 2b, with the difference that in this example, the elastic element (300) is of a tubular shape instead of a disc shape.

FIG. 3a discloses a cross sectional view of an elastic element. In this example, the elastic element (300) consists of an elastic thin walled tube. The elastic element has a first portion (301) for receiving the stoma and a second portion (311) for connecting the element to the device. The first portion of the elastic element (301) is the part of the tube which remains tubular after elastic element has been stretched. The second portion (311) of the elastic element is the stretched part of the tubular shaped elastic element.

FIG. 3b discloses a frontal view of the tubular elastic element (300). The hole (305) in the tubular elastic element (300) is adapted to receive the stoma of the human being. In FIGS. 3a and 3b, the second portion of the elastic element is not stretched and can therefore not be seen on drawing 3b.

FIG. 3c discloses a cross sectional view of the elastic element from FIG. 3a-b, comprising a first portion (301) and a second portion (311), but now the second portion (311) has been stretched. Similarly FIG. 3d discloses a frontal view of the elastic element from FIG. 3a-b, comprising a first portion (301) and a second portion (311), where the second portion (311) has been stretched. Consequently the stretched form of the elastic element comprises a tubular shaped part (the first portion, 301) and a disc shaped part (the second portion, 311). The hole (305) in the elastic element is adapted to receive the stoma. It should be noted that the elastic element is arranged such that after it has been stretched, the hole in the elastic element is still slightly smaller than the stoma.

It should also be noted that it has surprisingly been found that when a tubular element made from a flexible material is stretched outwardly by applying a force to the circumference of one end of the tubular element, then the tubular element assumes the form shown in FIGS. 3c and 3d. In other words it assumes a configuration where there is a stretched flange portion and an unstretched tubular portion. The stiffer the material, the more pronounced will be the distinction between the stretched portion and the unstretched portion. Also, the thicker the material, the more pronounced will be the distinction between the stretched portion and the unstretched portion.

FIG. 4a-b disclose views of a device comprising the elastic element described in example 3.

FIG. 4a discloses a cross sectional view of a device (100), comprising the elastic element (300) with a first portion (301) for receiving and contacting the stoma and second portion (311) for attaching the elastic element to the device. The device also comprises an attachment element (400) for attaching the device to a human being. The attachment element (400) is identical to the attachment element disclosed in FIG. 2. The device also comprises a stretching element (200) for stretching the second portion of the elastic element (311). The stretching element (200) is made of a rigid or semi rigid ring formed material. It should be noted that in this embodiment, the stretching element does not actively stretch the elastic element, rather, the elastic element is first stretched manually and then attached to the stretching element which maintains the stretched configuration of the elastic element.

FIG. 4b discloses a frontal view of the device (100), comprising an elastic element (300) with a first portion (301) and a second portion (311). The first portion of the elastic element (301) is at the circumference of the hole (305) for receiving the stoma. In this example, as shown on FIG. 4a, the first portion of the elastic element (301) is not stretched. The device comprises a ring formed stretching element (200) for stretching a portion of elastic element. In this example, the second portion of the elastic element (311) is fixed to the stretching element and is held in its stretched condition by the stretching element (200). The attachment element (400) of this example is a skin friendly adhesive, which is attached to the ring formed stretching element. In this example, the diameter of the most stretched part of the second portion of the elastic element (311) is stretched to a diameter being 3 times larger than the diameter when not being stretched.

While not shown in the figures, the elastic element could also be arranged as an element having two distinct portions, a first portion which is formed of a first elastic material in the form of a tubular element and a second portion which is formed as a disc shaped element formed from a second material. When attached to the stretching element, the second portion formed as a disc shaped element would be stretched, similar to the embodiment shown in FIGS. 2a-2b. The first and second materials could be the same material, or they could be different materials. In one embodiment the first material is softer than the second material. In another embodiment the first material is thinner than the second material.

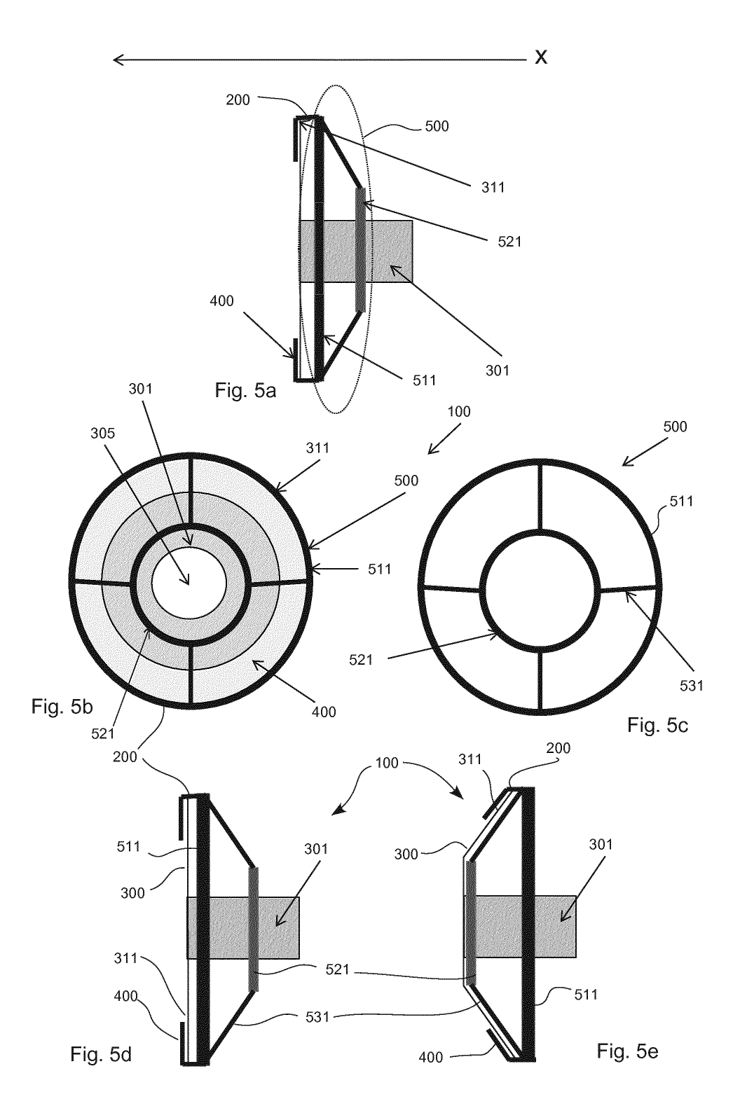

FIG. 5a-5e disclose views of a device (100) and an adjusting element (500) for adjusting the position of at least a portion of the elastic element relative to the attachment element.

FIG. 5a discloses a cross sectional view of a device (100), comprising an elastic element (300) of a tubular shape with a first portion (301) for receiving the stoma and a second portion (311) for connecting the elastic element to the device. The device also comprises an attachment element (400) for attaching the device to a human being. The adjusting element (500) comprises an anchoring portion (511) and a movable portion (521). The anchoring portion (511) of the adjusting element (500) is connected to the stretching element (200), which again is attached to the attachment element (400). The second portion of the elastic element (311) is attached to the stretching element (200). The movable portion of the adjusting element (521) is movable in relation to the anchoring portion (511) and consequently also in this example in relation to the stretching element (200) and the attachment element (400) in the x-direction as demonstrated on FIGS. 5d and 5e, which is intended to be approximately perpendicular to the peri-stomal skin.

FIG. 5b discloses a frontal view of the device (100). The adjusting element (500) comprises two rings—the anchoring portion (511) and the movable portion (521).

FIG. 5c discloses a frontal view of solely the adjusting element (500) in FIGS. 5a to 5e. The adjusting element (500) comprises an anchoring portion (511) and a movable portion (521). The movable portion is connected to the anchoring portion (511) via four arms (531). In this example, the four arms are longer than the difference in radius between the anchoring portion (511) and the movable portion (521) dictating a 3-dimensional structure of the adjusting element (as seen on FIG. 5a.

FIG. 5d discloses a cross sectional view of the device (100), comprising the elastic element (300) and the attachment element (400). The movable portion (521) of the adjusting element (500) is in its first disengaged position, where it does not engage with a portion of the elastic element (300).

FIG. 5e discloses a cross sectional view of the device (100) similar to 5d, but where the movable portion (521) of the adjusting element (500) has engaged with a portion of the elastic element (300) and moved the first portion of the elastic element in relation to the anchoring portion (511) in the x-direction. Overall, the adjusting element of this example has forced the elastic element to engage in a more inwards position on the stoma of the human being.

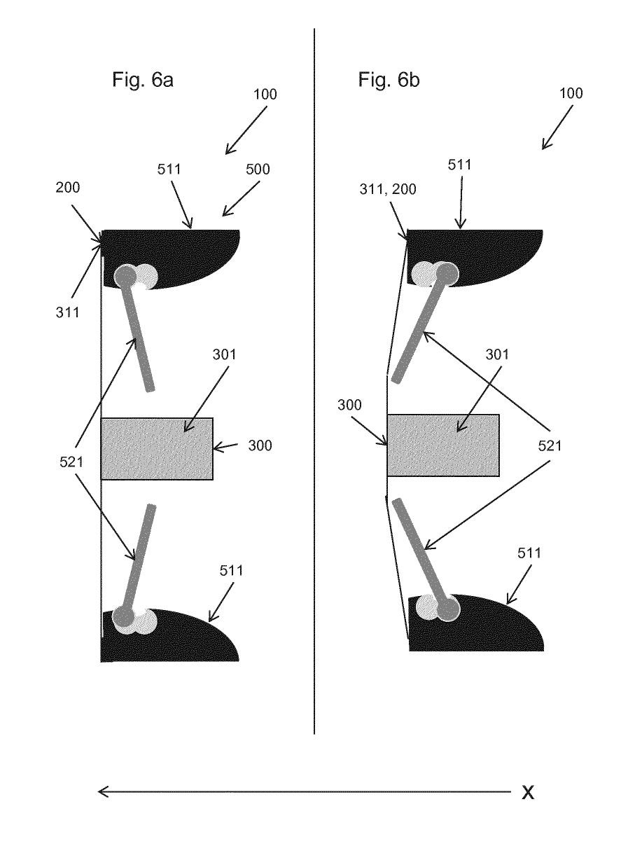

FIG. 6a-b disclose views of a different device (100) comprising an adjusting element (500) and an elastic element (300) according to the present invention. The elastic element (300) with a first portion (301) for receiving the stoma and second portion (311) connecting the elastic element to the device is of a tubular shape. The adjusting element (500) comprises an anchoring portion (511) and a movable portion (521). In this example, the movable portion is 4 discrete arms, which each have a first position and a second position. The elastic element is welded onto the adjusting element, the welding acting as the stretching element (200). The stretching element of the device is therefore integrally formed with the adjusting element.

FIG. 6a discloses a cross sectional view of the example of a device (100), where the movable portion (521) is in its first position, where it does not engage with a portion of the elastic element (300).

FIG. 6b discloses a cross sectional view of the example of a device (100), where the movable portion (521) is in its second position, where it has engaged with a portion of the elastic element (300) and moved a portion of the elastic element in relation to the anchoring portion (511) in the x-direction. It should be noted, that the movable portion (521) of the adjusting element engages with a portion of elastic element (300), which is stretched.

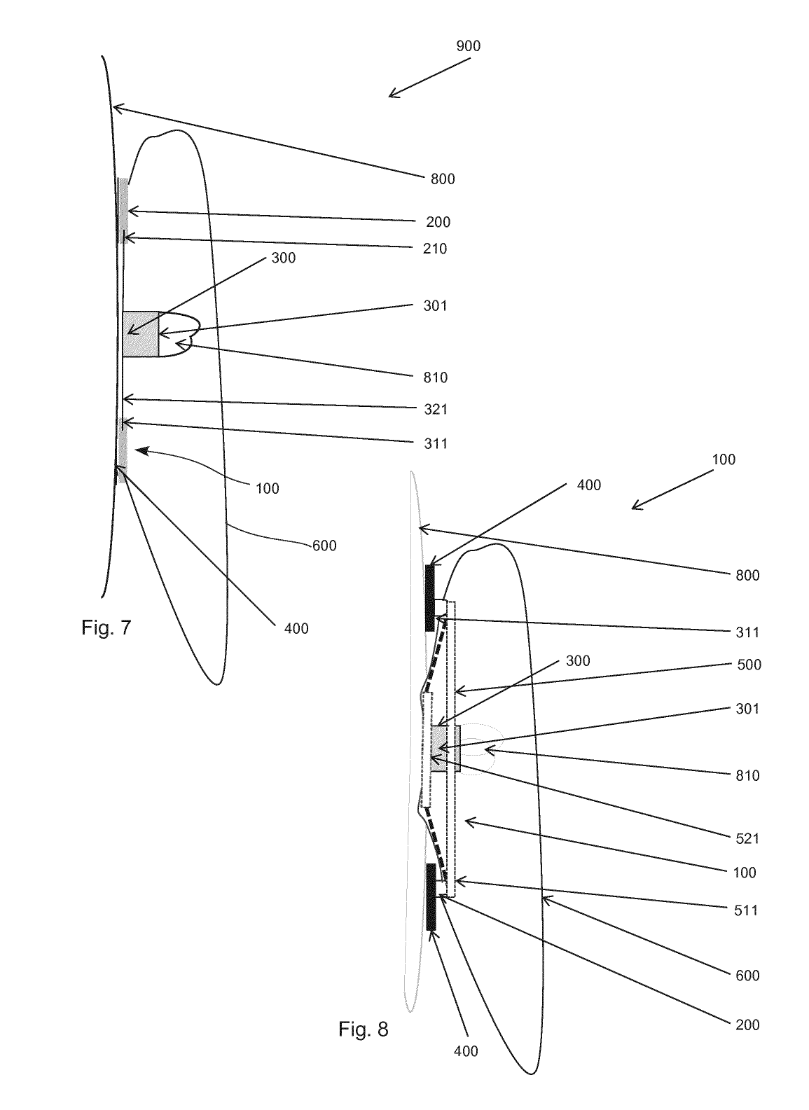

FIG. 7 discloses a cross sectional view of an example of a system (900) for directing and collecting output from a stoma, wherein the system comprises a device (100) similar to the device described on FIG. 3, the device (100) being attached to a human being (800). The system furthermore comprises a container (600) for collecting the output of the stoma. The elastic element (300) has a first portion (301) for receiving the stoma and a second portion (311) for attaching the elastic element to the device (100). The first portion (301) receives the stoma (810) and creates a seal against the outer circumference of the stoma (810). The second portion (311) attaches the elastic element (300) to the stretching element (200), and thereby stretches a portion of the elastic element. The second portion of the elastic element (311) is attached to the stretching element via a fastening mechanism (210). The device and system are attached to the human being via the attachment element (400), which in this example is a hydrocolloid adhesive. The hydrocolloid adhesive (400) is attached to the stretching element (200). The device comprises an ostomy bag (600) for collecting the output from the stoma (810). The ostomy bag (600) is attached to the stretching element (200). The stretched portion of the elastic element (321) supports the first portion of the elastic element (301) in staying in an inwards position on the stoma (810) during the wear time of the system.

FIG. 8 discloses a cross sectional view of an example of system (900) for directing and receiving output from a stoma comprising a device (100) similar to the device described on FIG. 5, the device being attached to the human being (800), allowing the first portion (301) of the elastic element (300) of the device (100) to receive and contact the circumference of the stoma (810). The elastic element (300) has a second portion (311) for attaching the elastic member to the device—in this figure attached to the stretching element (200). The first portion (301) is creating a seal against the outer circumference of the stoma (810). The adjusting element (500) according to FIG. 5, comprising an anchoring portion (511) and a movable portion (521), is also connected to the stretching element (200). The anchoring portion (511) is attached to the stretching element (200). In this disclosed drawing, the movable portion (521) of the adjusting element (500) has engaged with a portion of the elastic element (300) and moved the first portion of elastic element (301) in relation to the anchoring portion (511) in the x-direction ensuring a more inward position of the first portion of the elastic element (301) on the stoma (810). The combination of the position of the movable portion (521) and the elasticity of the stretched portion of the elastic element holds the first portion (301) of the elastic element in place on the stoma (810). The device (100) is attached to the human beings skin via the attachment element for attaching the device to the human being (400). The attachment element is a silicone adhesive with a film backing, the film backing being welded onto the stretching element. The system comprises an ostomy bag (600) for collecting the output from the stoma. The ostomy bag (600) is also coupled to the stretching element (200) via a coupling arrangement.

It can be noted that in the embodiment of FIG. 8, the adjusting element comprises an anchoring portion and a moveable portion. However, in another embodiment, it could be imagined that the adjusting element was fixed in the position shown in FIG. 8. In other words, the anchoring portion and the moveable element were fixed in position in relation to each other. This would provide for a more simple device without the ability to adjust the position of the adjusting element, but would in many cases suffice.

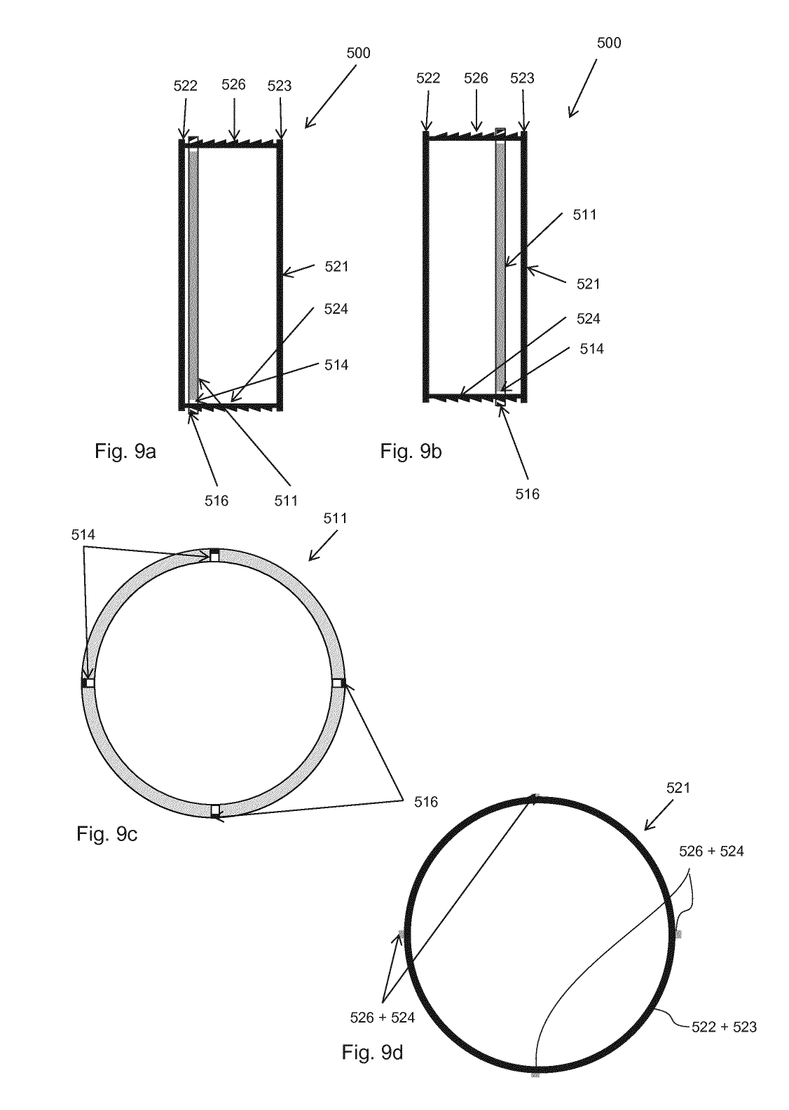

FIG. 9a-9d disclose views of another adjusting element (500) according to the present invention.

FIG. 9a discloses a cross sectional view of an adjusting element (500), comprising an anchoring portion (511) and a movable portion (521). The movable portion (521) comprises a front ring (522) and a back ring (523). The front ring (522) may engage with a stretched portion of an elastic element. The movable portion (521) also comprises arms (524) for positioning the movable portion relative to the anchoring portion (511). The arms (524) have multiple teeth (526) for engagement and positioning of the movable portion (521) relative to the anchoring portion (511). The anchoring portion (511) is a ring with receivers (514) for receiving the arms (524) of the movable portion (521). The anchoring portion (511) has a tooth portion (516) shaped to interact with the teeth (526) on the arm (524) of the movable portion (521). In this example the form of the teeth (526) and the form of the tooth (516) allow the movable portion to only move inwards and avoids any outwards movement of the movable portion (521).

FIG. 9b discloses a cross sectional view of the adjusting element (500) from FIG. 9a, wherein movable portion (521) has moved to a more inwards position relative to the anchoring portion (511).

FIG. 9c discloses a frontal view of the anchoring portion (511) from FIGS. 9a and 9b. The anchoring portion (511) comprises receivers (514) and each receiver comprises a tooth (516).

FIG. 9d discloses a frontal view of the movable portion (521) from FIGS. 9a and 9b. The movable portion (521) comprises rings (522+523) and arms with teeth (524+526).

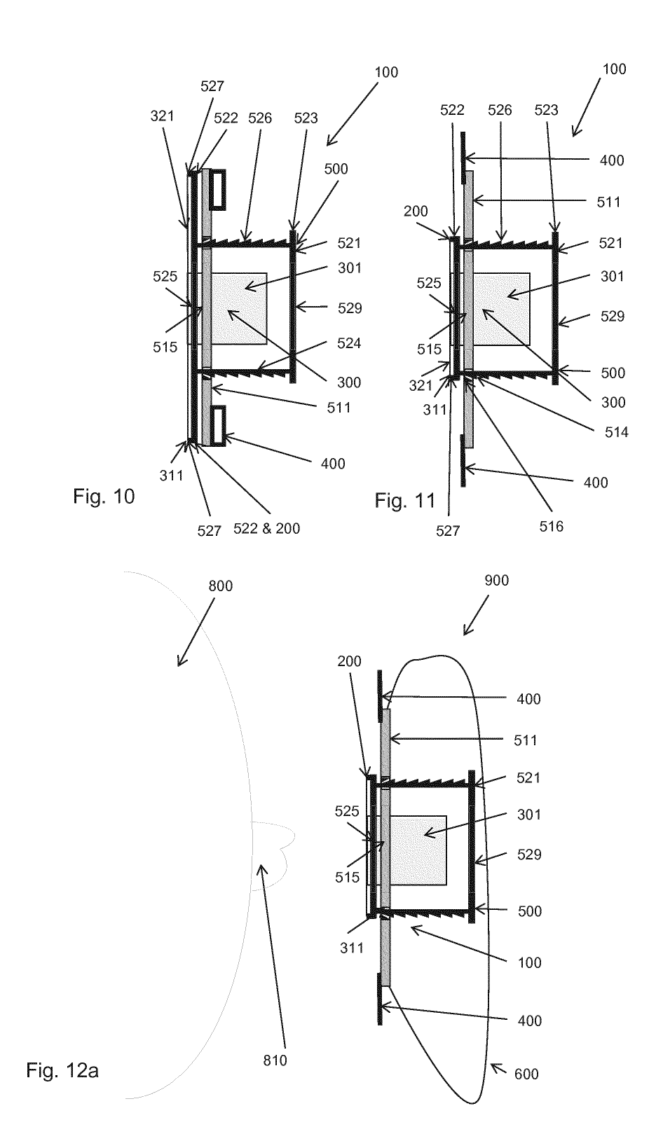

FIG. 10 and FIG. 11 disclose views of two different devices (100) for directing output from a stoma according to the present invention. The devices comprise an adjusting element (500) and a stretching element (200), which is integrated into the adjusting element (550). Furthermore the devices comprise a tubular shaped elastic element (300) as shown on FIG. 2 and an attachment element (400). In FIGS. 10 and 11, the described adjusting elements (500) have moveable portions (521) and anchoring portions (511) as shown on FIG. 9. The adjusting element's movable portion (521) and the anchoring portion (511) has a mechanism similar to the mechanism disclosed in FIG. 9, connecting the two elements together via 4 holes (514) on the anchoring portion (511) and 4 arms (524) on the movable portion (521).

FIG. 10 discloses a cross sectional view of a device (100) for directing output from a stoma. The second portion of elastic element (311) is attached to the front ring (522) of the movable portion (521) of the adjusting element (500). The fastening arrangement (527) fastens the second portion (311) of the elastic element to the front ring (522). The front ring (522) acts as a stretching element (200), stretching a portion of the elastic element (321) including the second portion (311). The anchoring portion (511) is disc formed. The hole in the anchoring portion (515) and the hole in the front ring (525) are adapted to receive the first portion of the elastic element (301). Furthermore, the anchoring portion (511) has 4 smaller holes (514), which size is adjusted to receive the arms (524) of the movable portion (521). In this example, the front ring (522) is disc formed similar to the anchoring portion (511) and has a larger outer diameter than the back ring (523). The hole (525) in front ring (522), the hole (529) in the back ring (523) and the hole (515) in the anchoring portion (515) are of same size. The attachment element (400) for attaching the device to the human being is a structure adapted to fit to a belt.

FIG. 11 discloses a cross sectional view of a device (100) for directing output from a stoma. The second portion (311) of the elastic element (300) is attached to the front ring (522) of movable portion (521) of the adjusting element (500). The fastening arrangement (527) fastens the second portion (311) of the elastic element (300) to the front ring (522) of the movable portion (521). As in FIG. 10, the front ring (522) is the stretching element (200), stretching a portion of the elastic element (321) including the second portion (311). The mechanism for connecting the anchoring portion (511) to the movable portion (521) is identical to the mechanism describe in FIG. 10. In FIG. 11, the stretched portion (321) is less stretched than in FIG. 10. The attachment element for attaching the device to the human being (400) is an adhesive layer attached to the outer section of the anchoring portion (511).

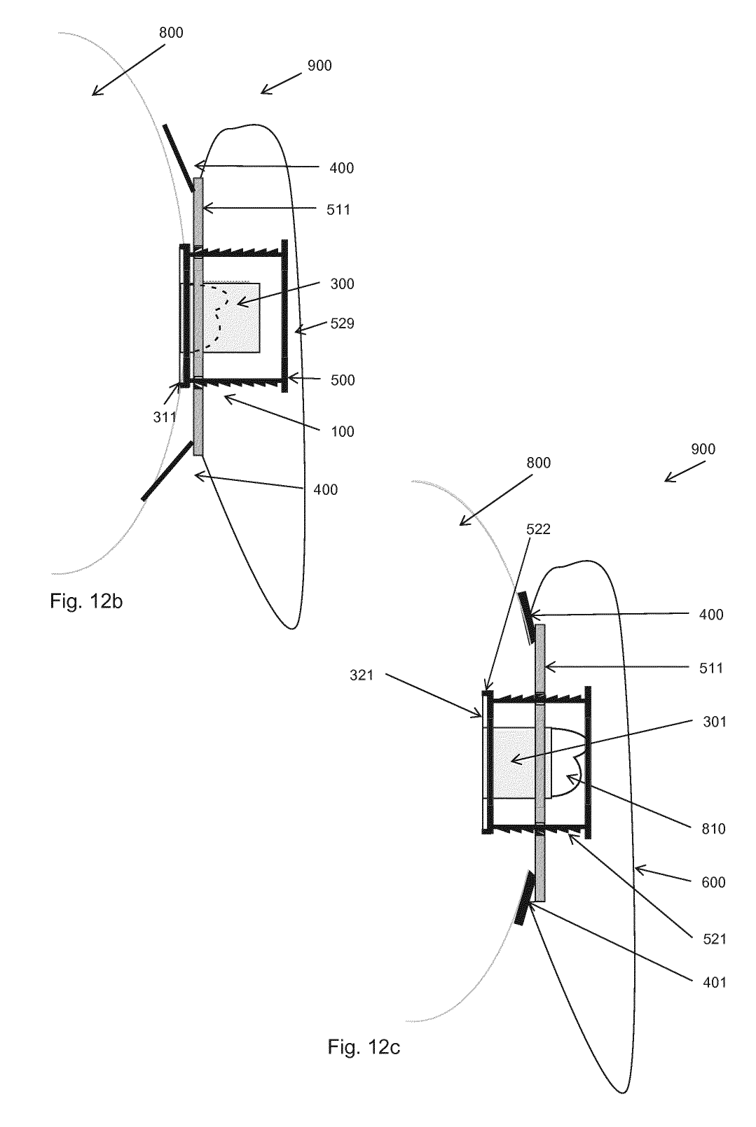

FIGS. 12a to 12c disclose cross sectional views of a system for directing and collecting output from a stoma (900) comprising a device (100) for directing output from a stoma (810) prior to application and during wear time. The system (900) and device (100) are examples according to the present invention. The system (900) comprises the device (100) described in FIG. 11.

FIG. 12a discloses a cross sectional view of the system (900) for directing and collecting output from a stoma (810) of a human being (800) just prior to attaching on the human being. The system comprises the device from example 11 and a container (600) for collecting the output, in this case an ostomy bag. The movable portion (521) of the adjusting element (500) is in its first position, where it has not moved a portion of the elastic element relative to the anchoring portion (511). The attachment element (400) is a silicone adhesive for adherence of the device onto the peri-stomal skin of the human being (800).

FIG. 12b discloses a cross sectional view of the system (900) from FIG. 12a, where the device is attached to the human being (800) by adhering the attachment element (400) to the peri-stomal skin. The movable portion (521) of the adjusting element (500) has not been moved relative to the anchoring portion (511).

FIG. 12c discloses a cross sectional view of the system (900) from FIG. 12a, where the device is attached to the human being (800) by adhering the attachment element (400) to the peri-stomal skin. The movable portion (521) of the adjusting element has been moved to a more inwards position relative to the anchoring portion (511). In this more inwards position, the movable portion (521) has moved the first portion of the elastic element (301) to a more inwards position in relation to the anchoring portion (511) and the stoma (810), as the elastic element is attached to the front ring (522) of the movable portion (521). Please note, that in this example, the portion of the elastic element that is moved by the movable portion is the entire elastic element. As a consequence of the position of the movable portion (521) and the front ring (522) relative to the anchoring portion (511) of the adjusting element (500) and as a consequence of the stretched portion (321) of the elastic element, the first portion of the elastic element will remain in place in an inwards position on the stoma (810) during wear time.

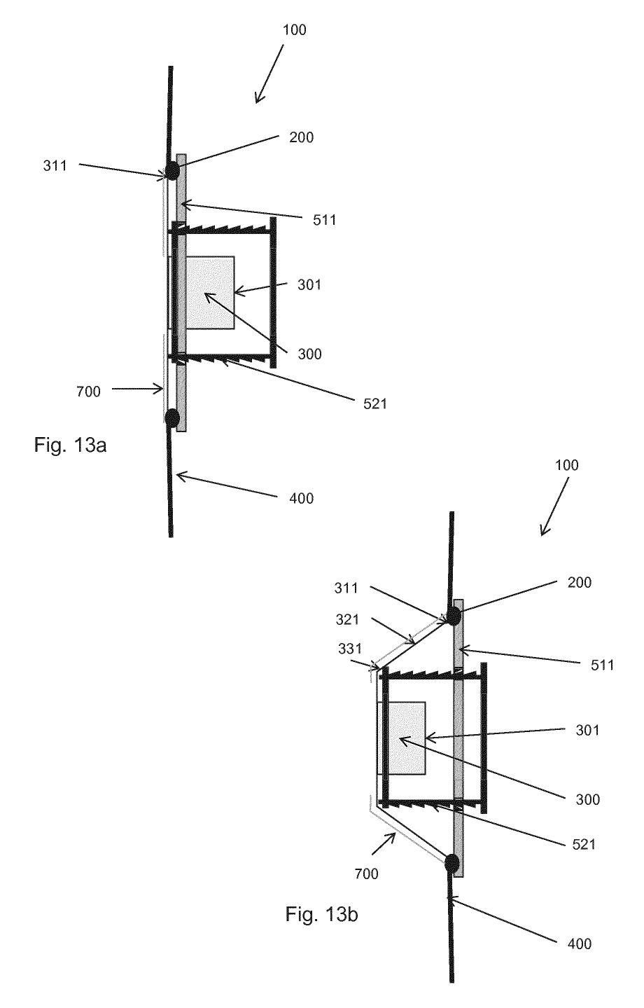

FIGS. 13a and 13b disclose cross sectional views of a device (100) for directing output from a stoma of a human being. The device (100) comprises an adjusting element comprising an anchoring portion (511) and a movable portion (521), an elastic element (300), a stretching element (200) and an attachment element (400) for attaching the device to the human being. The device has an adjusting element similar to the device in FIG. 11. The attachment element comprises a polyurethane adhesive for adhering the device to the peri-stomal skin. In this example, the movable portion (521) is separated from the stretching element (200), as the stretching element (200) is connected to the anchoring portion (511) of the adjusting element. The second portion of the elastic film (311) is attached to the stretching element (200). The device also comprises a skin contacting element (700), which in this example is a breathable non-woven layer.

In FIG. 13a, the movable portion (521) of the adjusting element is in its first position, where it does not move a portion of the elastic element relative to the anchoring portion (511) and the attachment element (400).

In FIG. 13b, the movable portion (521) of the adjusting element (500) is in a second position, where it does move a portion of the elastic element relative to the anchoring portion (511) and the attachment element. It should be noted, that in this example, the more inwards the movable portion (521) is moved relative to the anchoring portion (511), the portion of the elastic element (331) that contacts the movable portion becomes more distant from the second portion of the elastic element. As a consequence, the stretched portion (321) of the elastic element increases in size.

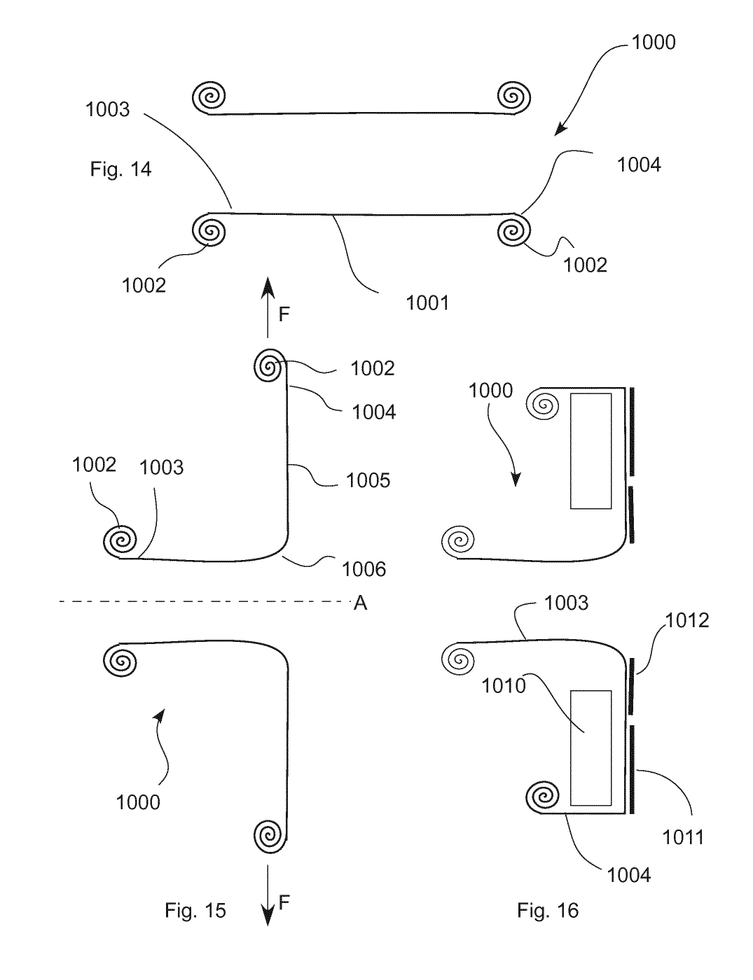

FIG. 14 shows a schematic representation of an embodiment 1000 of an elastic element. In this embodiment, the elastic element is in the form of a tubular element 1001 made from a silicone foil material. In this embodiment, both ends of the tubular element have been rolled up 1002. In the figures, the rolled up portions have been exaggerated for the sake of understanding, however, it should be clear to the person skilled in the art that in a real embodiment, the rolled up portions would be tightly rolled up to provide a small increase in thickness and stiffness at the ends of the tubular element. This makes the ends of the tubular element easier to manipulate. Furthermore at the stoma contacting side, the rolled up portion will in certain cases provide an extra sealing effect due to the extra stiffness and/or due to the “edge” which the rolled up portion creates. At the end of the second portion, the rolled up portion can be used to connect the second portion to the stretching element in a more secure manner.

In one embodiment (not shown) an adhesive could be arranged in the rolled up portion to make sure the rolled up portion does not unroll. In other embodiments, it could be considered that only one end of the tubular portion was rolled up, or neither was rolled up.

According to the current invention, the tubular portion has a first portion 1003 and a second portion 1004.

FIG. 15 schematically shows the elastic element of FIG. 14 and shows how the second portion of the tubular element 1000 of FIG. 14 has been stretched outwardly by applying a force to the rolled up portions perpendicular to the axis (A) of the tubular portion. The direction of the force is shown in FIG. 15 with the arrows F. The force is if possible applied uniformly around the circumference of the second portion, however, the force could also be applied at a number of discrete points in certain embodiments. As can be seen from FIG. 15, as the force is applied, the second portion is pulled outwardly and forms a flange portion 1005. In the flange portion 1005, the elastic foil material is in a stretched configuration. Due to the stretched configuration of the flange portion, the first portion of the elastic element 1003 is better able to adapt to the movements of the stoma while still maintaining a soft contact with the stoma.

The stretched configuration of the flange element ensures that the inward end 1006 of the first portion, or the transition area 1006 between the first portion and the second portion, assumes a “stiffer” position and if the transition area moves due to movement of the stoma, it will always seek back to its original position due to the stretch, or pre-stress, in the flange portion. This could be considered a form of “repositioning” effect.

In this way, the pre-stress allows the use of a softer material for the elastic material. In prior art devices with no pre-stress, a relatively thick foil material is needed in order to ensure that the seal is maintained during motion of the stoma. However, by using a pre-stressed (or pre-stretched) portion, the “location stiffness”, or repositioning effect, of the stoma contacting portion can be increased, without increasing the stiffness of the material of the elastic element itself. In this way, a good seal can be established while maintaining a soft connection between the stoma and the elastic element.

In order to further increase the repositioning effect of the elastic portion, in one embodiment, a material/substance used to decrease the friction between the first portion of the elastic element and the stoma could be applied to the inner side of the first portion of the elastic element. For example an oil, for example corn oil, could be applied to the inner side. Due to the friction reducing substance, the first portion of the elastic element would be able to slide on the stoma and reposition itself due to the force applied by the stretched portion of the elastic element.

FIG. 16 shows how the second portion 1004 of the elastic element 1000 has been wrapped around an annular ring shaped element 1010. In this way, the annular ring shaped element 1010 acts as a stretching element and maintains a portion of the elastic element in a stretched configuration. An attachment element 1011 in the form of a two sided adhesive tape is shown schematically in the figure. Also a skin contacting element 1012 is also shown schematically in the figures. The skin contacting element is an absorbent fibre based material attached to the elastic element via an adhesive. The skin contacting element increases the wear comfort of the device.

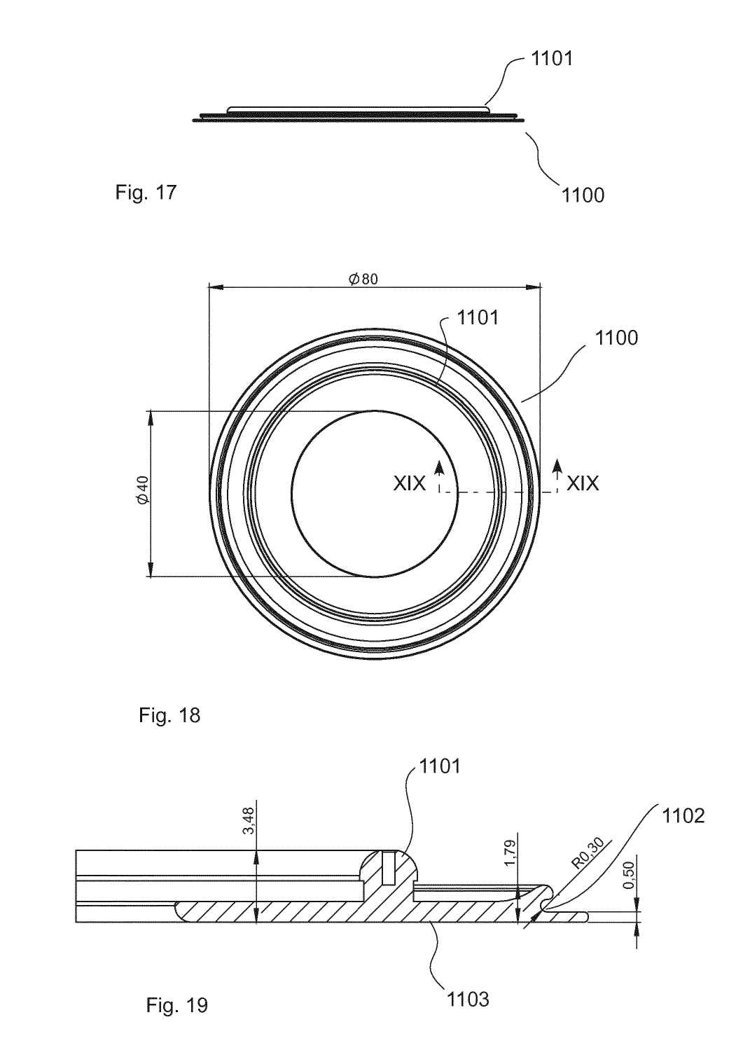

FIGS. 17-19 show an embodiment of a concrete example 1100 of a stretching element according to the current invention. The stretching element 1100 is a ring shaped element formed as an injection moulded component. The stretching element has a connection element 1101 for connecting it to an ostomy pouch. The stretching element also comprises a receiving element 1102 into which the second portion of an elastic element can be arranged. The bottom surface 1103 of the stretching element will be arranged facing the skin of the human being, once the device is attached to the human being. Some dimensions are shown on the figures in order to give an indication of possible dimensions; however the dimensions should not be limiting to the scope of protection of the current invention.

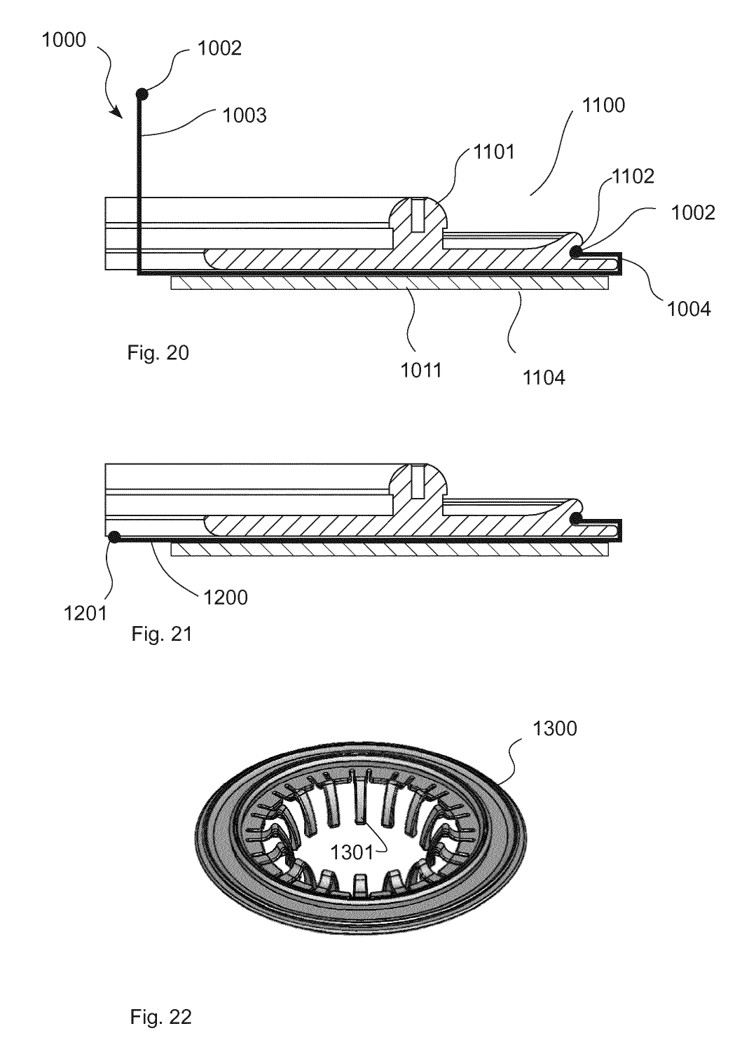

FIG. 20 shows in schematic cross section an example of an elastic element 1000 in the form of a tubular element mounted on the stretching element of FIGS. 17-19. Note that only the right side of the device is shown. An attachment element 1011 is attached to the skin facing surface of the elastic element. The skin facing surface 1104 of the attachment element 1011 is considered the “first surface” for the sake of this specification.

FIG. 21 shows in schematic cross section an example of an elastic element 1200 in the form of a disc mounted on the stretching element 1100 of FIGS. 17-19. Again note that only the right side of the device is shown. In this embodiment, instead of a tubular element, the elastic element is purely a disc, similar in shape to a CD though smaller in size. Instead of a tubular first portion, the first portion 1201 in this embodiment is purely the inside edge of the disc shaped element.

FIG. 22 shows an example of a stretching element 1300 which comprises a number of protrusions 1301 which together function as an integrated adjusting element which press the elastic element in towards the body of the human to which the stretching element is applied. The protrusions are formed slightly elastic to ensure that the protrusions can respond to movements of the body.

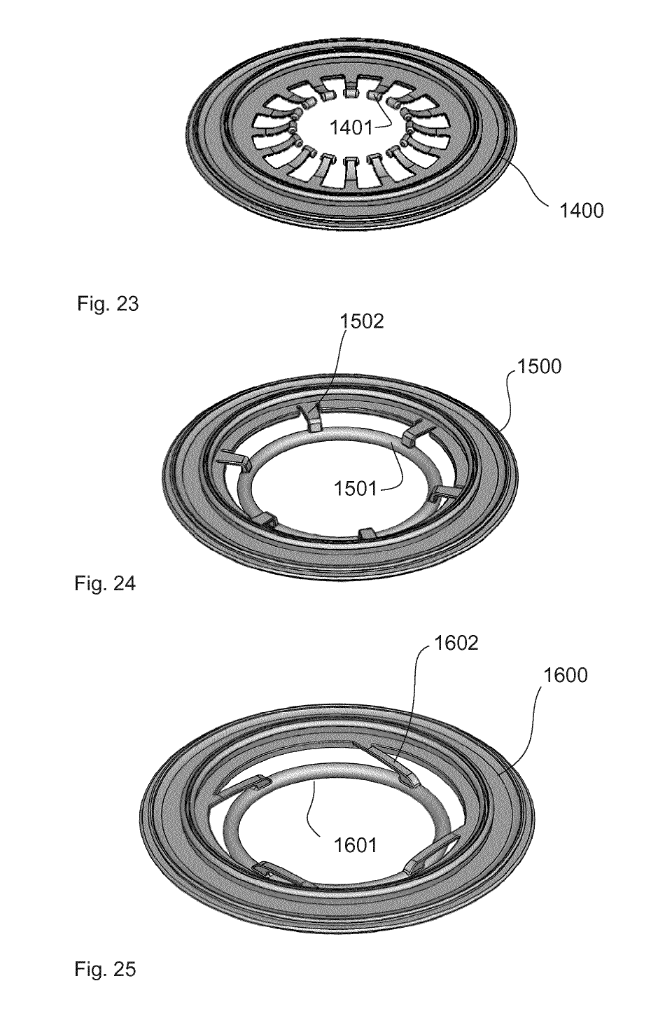

FIG. 23 shows another example of a stretching element 1400 which again comprises a number of protrusions 1401 which function as an adjusting element. In the figure, the protrusions are shown bent outwardly to show that they are able to adapt to the movements of the body. However, in normal state, they would be bent in towards the skin of the human, ie in a direction opposite to the one shown in the figures.

FIG. 24 shows another example of a stretching element 1500 which comprises a ring shaped element 1501 as an adjusting element. The ring shaped element 1501 is attached to the stretching element via four short arms 1502. The short arms are slightly flexible to allow a certain amount of flexibility.

FIG. 25 shows another example of a stretching element 1600 which is very similar to the one shown in FIG. 24, but is more flexible than the one of FIG. 24 since the arms 1602 which connect the adjusting element 1601 to the stretching element are arranged tangent to the circumference of the stretching element and can therefore be made longer than the arms of the embodiment of FIG. 24.

It should be noted that the embodiment of the stretching element of FIGS. 17-19 would be suitable for use with a stoma which is protruding from the body. The embodiments of the stretching elements of FIGS. 22-25 would be especially suitable for use with a stoma which is not protruding from the body or which is not protruding very far. Furthermore, it can be noted that for different stomas, the amount the adjusting element is offset from the first surface of the attachment element, or from the skin facing side of the stretching element, can be increased or decreased as required.

1. A device for directing output from a stoma, the device comprising:
at least one elastic element, said elastic element comprising a first portion having a hole for receiving and contacting the stoma and a second portion for attaching the elastic element to one of the other elements of the device;
a stretching element for stretching at least a portion of the elastic element in a direction which is parallel to the plane of the hole in the first portion; and
an attachment element for attaching the device to the human being, the attachment element comprising a first surface which is a portion of the attachment element which is either in direct contact with or which is closest to the peri-stomal skin when the device is attached to the human being.

2. A device according to claim 1, wherein the first portion of the elastic element is of a tubular shape.

3. A device according to claim 1, characterized in that the elastic element is a tubular element.

4. A device according to claim 3, characterized in that the elastic element has a uniform diameter.

5. A device according to claim 1, characterized in that the elastic element is a planar element in the shape of a disc.

6. A device according to claim 1,
characterized in that the elastic element is made from an elastic foil material having a uniform thickness.

7. A device according to claim 1, characterised in that the device further comprises a receiving element for receiving and holding the second portion of the elastic element without damaging the elastic element.

8. A device according to claim 1, characterized in that prior to attaching the device to a human being, a portion of the elastic element has been stretched to become a stretched portion and is maintained in its stretched configuration by the stretching element and in that the outer circumference of the stretched portion of the elastic element is at least stretched to a circumference 40% larger than the circumference of the portion when not stretched.

9. A device according to claim 1, characterized in that the device comprises an adjusting element for adjusting the position of at least a portion of the elastic element relative to the attachment element in at least a direction normal to the first surface of the attachment element.

10. A device according to claim 9, wherein the adjusting element comprises an anchoring portion and a movable portion.

11. A device according to claim 10, wherein the movable portion of the adjusting element is adapted to be positioned in at least 3 different positions relative to the anchoring portion.

12. A system for directing and collecting output from a stoma, the system comprising:
a device according to claim 1; and
a suitable container for collecting the output from the stoma.

13. A system according to claim 12, characterized in that said container is detachably connected to said device.

14. A system according to claim 12, characterized in that said container is integrally attached to said device.

15. A method for providing a device for directing output from a stoma of a human being, the method comprising the steps of:
providing an elastic element with a first and second portion;
providing a stretching element;
providing an attachment element;
expanding the second portion of the elastic element prior to attaching the device to the human being;
connecting the second portion of the elastic element to the stretching element to maintain a portion of the elastic element in a stretched configuration prior to attaching the device to the human being; and
connecting the attachment element to the elastic element and/or the stretching element.
